(19) United States
(12) Patent Application Publication
(10) Pub. No.: US 2026/0028584 A1
(43) Pub. Date: Jan. 29, 2026

(54) METHOD FOR EVALUATING AGGREGATION-SUPPRESSING ACTIVITY OR AGGREGATION-PROMOTING ACTIVITY ON AGGREGATING PROTEIN USING ORGANOID, AND METHOD FOR PRODUCING ORGANOID

(71) Applicants: KANEKA CORPORATION, Osaka (JP); MURORAN INSTITUTE OF TECHNOLOGY, Hokkaido (JP)

(72) Inventors: Naoki Nishishita, Hyogo (JP); Kiyotaka Tokuraku, Hokkaido (JP); Koji Uwai, Hokkaido (JP); Shinya Watanabe, Hokkaido (JP); Masahiro Kuragano, Hokkaido (JP)

(73) Assignees: KANEKA CORPORATION, Osaka (JP); MURORAN INSTITUTE OF TECHNOLOGY, Hokkaido (JP)

(21) Appl. No.: 19/346,263

(22) Filed: Sep. 30, 2025

Related U.S. Application Data
(63) Continuation of application No. PCT/JP2024/080040, filed on Mar. 29, 2024.

(30) Foreign Application Priority Data
Mar. 30, 2023 (JP) 2023-054595
Mar. 30, 2023 (JP) 2023-054650

(51) Int. Cl. C12N 5/0793; G01N 33/58

(52) U.S. Cl. CPC C12N 5/0619; G01N 33/588; C12N 2503/00; C12N 2506/45

(57) ABSTRACT

The present invention aims to provide a method for screening a substance having an aggregation-suppressing activity or aggregation-promoting activity on an aggregating protein, and a method for producing an organoid and an organoid produced by this method. Specifically, the present invention relates to a method for evaluating an aggregation-suppressing activity or aggregation-promoting activity of a test substance on an aggregating protein, the method including allowing an aggregating protein labeled with a label, a test substance, and an organoid to coexist in an aqueous solution, and quantifying the aggregating protein aggregated and/or deposited on a surface of the organoid and/or inside the organoid using the label as an index; and a method for producing an organoid, including forming a spheroid by culturing a cell, embedding the obtained spheroid in a gel, and culturing the embedded spheroid in the gel.

### TECHNICAL FIELD

One or more embodiments of the present invention relates to a method for evaluating an aggregation-suppressing activity or aggregation-promoting activity on an aggregating protein using, for example, an organoid, and a method for producing an organoid and an organoid produced by this method.

### BACKGROUND

Alzheimer's disease (often abbreviated herein as “AD”) is a type of irreversible progressive central nervous system disease, which is accompanied by symptoms such as cognitive impairment (dementia), behavioral disorders, or personality changes. The number of dementia patients worldwide is estimated to be 50 million or more as of 2019, but about 70% are considered to be AD patients, and the incidence rate thereof tends to increase. In recent years, an increase in medical expenses and care problems due to an increase in the number of AD patients have become major social problems such as economic and mental burdens on countries and those concerned with patients.

AD begins with aggregation and accumulation of amyloid β protein (herein often referred to as “Aβ”), which is a hydrophobic peptide, in the brain of a patient. Then, tau protein, which is a microtubule-associated protein, is hyperphosphorylated and fibrillated, and then neuronal cells are destroyed and the brain atrophies, whereby AD develops (NPLs 1 to 3).

Based on this onset mechanism of AD, techniques have been developed for evaluating the Aβ aggregation inhibitory effect of test substances added in vitro and screening candidate compounds for AD treatment. For example, microliter-scale high-throughput screening (herein referred to as “MSHTS”) of amyloid β protein aggregation inhibitors using quantum-dot nanoprobes is a screening technique based on a Cell-free Assays (cell-free test) system that can search for candidate compounds having an inhibitory effect on the aggregation of amyloid β proteins in PBS medium (NPL 4).

As an example of MSHTS, for example, PTL 1 discloses a method, an apparatus, and a program for evaluating amyloid formation, and specifically discloses a method for determining the amyloidogenic inhibitory activity of a test substance comprising an aggregation reaction step of reacting an amyloidogenic protein such as amyloid β protein with a fluorescent probe (containing a quantum dot as a fluorescent dye or a quantum dot or the like) capable of binding to amyloid formed by polymerization of the amyloidogenic protein in PBS in the presence or absence of a test substance; an image capturing step of capturing an image of fluorescence of the aggregation reaction product obtained in the aggregation reaction step; a standard deviation calculation step of calculating standard deviations of luminance values of pixels included in a region of interest in the fluorescence image captured in the image capturing step; and an activity determination step of comparing the standard deviation value of the luminance values in the presence of the test substance calculated in the standard deviation calculation step with the standard deviation value of the luminance values in the absence of the test substance and determining the test substance as having amyloidogenic inhibitory activity when the standard deviation value of the luminance values in the presence of the test substance is smaller than the standard deviation value of the luminance values in the absence of the test substance.

In addition, PTL 2 discloses a general-purpose quantum dot nanoprobe for evaluating the amyloid aggregation property of a protein or peptide, and a method for evaluating an amyloidogenic inhibitor using the quantum dot nanoprobe. Specifically, PTL 2 discloses a quantum dot nanoprobe in which a quantum dot is bonded to the N-terminus or C-terminus of an amyloidogenic peptide via cysteine.

However, in many cases, a candidate compound obtained in the Cell-free Assays system does not exhibit its inhibitory effect in the Cell-Based Assays (test using cells) system. This is considered to be because the Cell-free Assays system does not necessarily reflect the living body environment. In addition, in the Cell-Based Assays (test using cells) system, cultured cells in a two-dimensional environment cannot reproduce complex signal exchange between cells and an ECM (human extracellular matrix) and thus cannot be directly reflected in clinical tests (NPL 5). Therefore, organoids (three-dimensional tissue cells), which are cultured cells in a three-dimensional environment, can reproduce complex signal exchange between cells and an ECM, and are expected to be tissue cells closer to the physiological environment in vivo.

### PATENT LITERATURE

PTL 1: International Publication No. 2020/138265 PTL 2: Japanese Unexamined Patent Application Publication No. 2017-007990

### Non-Patent Literature

NPL 1: Hardy J. and Selkoe D. J., 2002, Science, 297 (5580): 353-356
NPL 2: Jack C. R. Jr., et al., 2010, Lancet Neurol. 9 (1): 119-128
NPL 3: Akira Tamaoka, 2017, Proceedings of the Annual Meeting of the Japanese Research Group on Senile Dementia, Vol. 22, No. 3, p. 19-23
NPL 4: Ishigaki et al., 2013, PLOS ONE, 8 (8): e72992
NPL 5: Antoni, Burckel, Josset and Noel, Int J Mol Sci., 2015, 16 (3): 5517-5527

### SUMMARY

In view of the above-described circumstances, an objective of one or more embodiments of the present invention is to provide a screening method capable of searching for a candidate compound having an aggregation-suppressing effect or an aggregation-promoting effect on an aggregating protein such as Aβ in an environment closer to in vivo using an organoid. Another objective of one or more embodiments of the present invention is to provide a method for producing an organoid that enables screening of a candidate compound useful for prevention or treatment of a disease in an environment closer to in vivo.

Thus, as a result of intensive studies to achieve the above objective, the present inventors have found that, as a test system using an organoid, an aggregating protein labeled with a label, a test substance, and an organoid are allowed to coexist, the aggregating protein aggregated and/or deposited on the surface of the organoid and inside the organoid is quantified using the label as an index, so that it is possible to evaluate whether or not the test substance is a substance having an aggregation-suppressing activity or an aggregation-promoting activity on the aggregating protein, thereby completing one or more embodiments of the present invention. The present inventors have also found that an organoid that can be used for screening of a candidate compound useful for prevention or treatment of a disease can be produced through a specific organoid production process, thereby completing one or more embodiments of the present invention.

That is, one or more embodiments of the present invention includes the following.
[1]A method for evaluating an aggregation-suppressing activity or aggregation-promoting activity of a test substance on an aggregating protein, the method including a step of allowing an aggregating protein labeled with a label, a test substance, and an organoid to coexist in an aqueous solution, and a step of quantifying the aggregating protein aggregated and/or deposited on a surface of the organoid and/or inside the organoid using the label as an index.
[2] The method according to [1], wherein the organoid is selected from the group consisting of a cerebral organoid, a cerebellar organoid, an inner ear organoid, a thyroid organoid, a thymus organoid, a T-cell matured lymphoid organoid, a cardiac muscle organoid, a lung organoid, a liver organoid, a pancreas organoid, a kidney organoid, a gastric gland organoid, a gut organoid, an epithelium organoid, an ovary organoid, a testis organoid, and a fused organoid including these organoids.
[3] The method according to [1] or [2], wherein the aggregating protein is selected from the group consisting of amyloid β protein, tau protein, α-synuclein protein, prion protein, huntingtin protein, amylin protein, apolipoprotein A1, serum amyloid A protein, immunoglobulin light chain, MAP4 protein, 32 microglobulin, TDP-43 protein, and cystatin C protein.
[4] The method according to any one of [1] to [3], wherein the label is an optical label.
[5] The method according to [4], wherein the optical label is a quantum dot.
[6] The method according to any one of [1] to [5], wherein the quantifying step includes a step of capturing an image of the organoid, and a step of calculating, as the aggregating protein aggregated and/or deposited on the surface of the organoid and/or inside the organoid, a ratio of an area of a fluorescent region from the label to a surface area of the organoid in the image obtained in the capturing step.
[7] The method according to any one of [1] to [6], wherein the quantifying is performed over time while the organoid is maintained.
[8] The method according to any one of [1] to [7], wherein the quantifying is performed for at least 7 days without using a genetically modified cell.
[9]A method for producing an organoid, including a step of forming a spheroid by culturing a cell for a culture time of 20 hours or more and 100 hours or less, a step of embedding the spheroid obtained in the spheroid forming step in a gel, and a step of culturing the spheroid embedded in the gel embedding step in the gel.
[10] The method according to [9], wherein the in-gel culturing step includes measuring a concentration of a cell migration protein in a culture supernatant.
[11] The method according to [9] or [10], wherein the organoid is selected from the group consisting of a cerebral organoid, a cerebellar organoid, an inner ear organoid, a thyroid organoid, a thymus organoid, a T-cell matured lymphoid organoid, a cardiac muscle organoid, a lung organoid, a liver organoid, a pancreas organoid, a kidney organoid, a gastric gland organoid, a gut organoid, an epithelium organoid, an ovary organoid, a testis organoid, and a fused organoid including these organoids.
[12] The method according to any one of [9] to [11], wherein the cell is selected from the group consisting of an induced pluripotent stem cell (iPS cell), a neural progenitor cell, a mesenchymal stem cell, and a cell differentiated from these cells.
[13] An organoid with an aggregated and/or deposited aggregating protein, including an organoid produced by the method according to any one of [9] to [12] and an aggregating protein aggregated and/or deposited on a surface of the organoid and/or inside the organoid.
[14] The organoid according to [13], wherein the organoid is maintained.

The disclosure of Japanese Patent Application Nos. 2023-054595 and 2023-054650 from which priority of the present application is claimed is incorporated herein by reference.

### BRIEF DESCRIPTION OF THE DRAWINGS

FIG. 1 is a photograph showing the influence of embryoid body (EB) preparation time on the formation of an organoid-like tissue structure in Example 1.

FIG. 2A is a photograph showing how cells infiltrate into and fill a gel in Example 1.

FIG. 2B is a graph showing changes in CCL2 concentration in a culture supernatant during in-gel culturing in Example 1.

FIG. 3 is a photograph showing how aggregation and deposition of Aβ on and in an organoid proceed over time in Example 2.

FIG. 4 shows the results of the Aβ deposition suppression effect of various test samples evaluated using an organoid in Example 3.

### DETAILED DESCRIPTION

One or more embodiments of the present invention will be described in detail below.

### 1. Method for Evaluating Aggregation-Suppressing Activity or Aggregation-Promoting Activity of Test Substance on Aggregating Protein

### 1-1. Overview

An aspect of one or more embodiments of the present invention is a method for evaluating the aggregation-suppressing activity or aggregation-promoting activity of a test substance on an aggregating protein. The method of this aspect includes a step of allowing an aggregating protein labeled with a label, a test substance, and an organoid to coexist in an aqueous solution, and a step of quantifying the aggregating protein aggregated and/or deposited on the surface of the organoid and/or inside the organoid using the label as an index. According to the method of this aspect, a candidate substance that exhibits an aggregation-suppressing activity or aggregation-promoting activity on an aggregating protein can be selected (screened).

### 1-2. Definitions

The following terms used in this specification will be defined. Unless otherwise specified, the following definitions described in this section are common to other aspects of one or more embodiments of the present invention.

### (1) Organoid

An organoid refers to a three-dimensional tissue-like structure produced by self-organization of cells and having a function similar to that of a tissue or an organ.

Examples of organoids include cerebral organoids, cerebellar organoids, inner ear organoids, thyroid organoids, thymus organoids, T-cell matured lymphoid organoids, cardiac muscle organoids, lung organoids, liver organoids, pancreas organoids, kidney organoids, gastric gland organoids (gastruloids), gut organoids such as foregut organoids (organoids constituting the mouth and stomach), midgut organoids (organoids constituting the small intestine and ascending colon), and hindgut organoids (organoids constituting the rectum and parts other than the ascending colon), epithelium organoids, ovary organoids, and testis organoids. Further examples include fused organoids that are created by fusing these organoids together to produce new differentiated cells.

### (2) Aggregating Protein

An aggregating protein refers to a protein whose molecules aggregate to form an aggregate. The property of the aggregate-forming protein may be liquid, sol, or solid, and the type of aggregating protein is not limited. Examples include polyglutamic acid, proteins associated with diseases, and autophagy-related proteins.

Aggregating proteins associated with diseases include, for example, amyloid β protein and tau protein (including phosphorylated tau protein) which are causative proteins of Alzheimer's disease, α-synuclein protein which is a causative protein of Parkinson's disease, prion protein which is a causative protein of transmissible spongiform encephalopathy (including Creutzfeldt-Jakob disease, mad cow disease, or prion disease), huntingtin protein which is a causative protein of Huntington's disease, amylin protein which is a causative protein of type II diabetes, apolipoprotein A1 (APOA1 protein) which is a causative protein of arterioscleroses (including cerebral infarction, lung infarction, and myocardial infarction), serum amyloid A protein which is a causative protein of rheumatoid arthritis, immunoglobulin light chain which is a causative protein of systemic AL amyloidosis, MAP4 protein which is a causative protein of myocardial infarction, 32 microglobulin which is a causative protein of dialysis amyloidosis, and TDP-43 protein considered to be a causative protein of amyotrophic lateral sclerosis. In particular, amyloid β proteins such as amyloid β40 protein, amyloid β42 protein, amyloid β43 protein, and amyloid β38 protein are particularly used.

Aggregating proteins (Main Protein misfolding/aggregating diseases) and diseases associated with the aggregating proteins (proteopathies) are shown in Table 1 below.

For example, α-synuclein, TDP-43, prion, cystatin C, and amyloid β are known as aggregating proteins, and they are respectively associated with Parkinson's disease, frontotemporal lobar degeneration, prion disease, chronic kidney disease (CKD), and Alzheimer's dementia or diabetic dementia.

### Table 1

1. Association of Major Aggregating Proteins with Proteopathies

| | Main protein misfolding/ |
|---|---|
| Proteopathy | aggregating diseases |
| Alzheimer's disease | Amyloid β peptide (Aβ); Tau |
| | protein (see tauopathies) |
| Cerebral β-amyloid | Amyloid β peptide (Aβ) |
| angiopathy | |
| Retinal ganglion cell | Amyloid β peptide (Aβ) |
| degeneration in glaucoma | |
| Prion diseases (multiple) | Prion protein |
| Parkinson's disease and | α-Synuclein |
| other synucleinopathies | |
| (multiple) | |
| Tauopathies (multiple) | Microtubule-associated |
| | protein tau (Tau protein) |
| Frontotemporal lobar | TDP-43 |
| degeneration (FTLD) (Ubi+, | |
| Tau−) | |
| FTLD-FUS | Fused in sarcoma (FUS) |
| | protein |
| Amyotrophic lateral | Superoxide dismutase, TDP- |
| sclerosis (ALS) | 43, FUS, C9ORF72, ubiquilin- |
| | 2 (UBQLN2) |
| Huntington's disease and | Proteins with tandem |
| other trinucleotide repeat | glutamine expansions |
| disorders (multiple) | |
| Familial British dementia | ABri |
| Familial Danish dementia | Adan |
| Hereditary cerebral | Cystatin C |
| hemorrhage with amyloidosis | |
| (Icelandic) (HCHWA-I) | |
| CADASIL | Notch3 |
| Alexander disease | Glial fibrillary acidic |
| | protein (GFAP) |
| Pelizaeus-Merzbacher disease | proteolipid protein (PLP) |
| Seipinopathies | Seipin |
| Familial amyloidotic | Transthyretin |
| neuropathy, Senile systemic | |
| amyloidosis | |
| Serpinopathies (multiple) | Serpins |
| AL (light chain) amyloidosis | Monoclonal immunoglobulin |
| (primary systemic | light chains |
| amyloidosis) | |
| AH (heavy chain) amyloidosis | Immunoglobulin heavy chains |
| AA (secondary) amyloidosis | Amyloid A protein |
| Type II diabetes | Islet amyloid polypeptide |
| | (IAPP; amylin) |
| Aortic medial amyloidosis | Medin (lactadherin) |
| ApoAI amyloidosis | Apolipoprotein AI |
| ApoAII amyloidosis | Apolipoprotein AII |
| ApoAIV amyloidosis | Apolipoprotein AIV |
| Familial amyloidosis of the | Gelsolin |
| Finnish type (FAF) | |
| Lysozyme amyloidosis | Lysozyme |
| Fibrinogen amyloidosis | Fibrinogen |
| Dialysis amyloidosis | Beta-2 microglobulin |
| Inclusion body | Amyloid β peptide (Aβ) |
| myositis/myopathy | |
| Cataracts | Crystallins |
| Retinitis pigmentosa with | rhodopsin |
| rhodopsin mutations | |
| Medullary thyroid carcinoma | Calcitonin |
| Cardiac atrial amyloidosis | Atrial natriuretic factor |
| Pituitary prolactinoma | Prolactin |
| Hereditary lattice corneal | Keratoepithelin |
| dystrophy | |
| Cutaneous lichen amyloidosis | Keratins |
| Mallory bodies | Keratin intermediate |
| | filament proteins |
| Corneal lactoferrin | Lactoferrin |
| amyloidosis | |
| Pulmonary alveolar | Surfactant protein C (SP · C) |
| proteinosis | |
| Odontogenic (Pindborg) tumor | Odontogenic ameloblast- |
| amyloid | associated protein |
| Seminal vesicle amyloid | Semenogelin I |
| Apolipoprotein C2 | Apolipoprotein C2 (ApoC2) |
| amyloidosis | |
| Apolipoprotein C3 | Apolipoprotein C3 (ApoC3) |
| amyloidosis | |
| Lect2 amyloidosis | Leukocyte chemotactic |
| | factor-2 (Lect2) |
| Insulin amyloidosis | Insulin |
| Galectin-7 amyloidosis | Galectin-7 (Gal7) |
| (primary localized cutaneous | |
| amyloidosis) | |
| Corneodesmosin amyloidosis | Corneodesmosin |
| Enfuvirtide amyloidosis | Enfuvirtide |
| Cystic fibrosis | cystic fibrosis |
| | transmembrane conductance |
| | regulator (CFTR) protein |
| Sickle cell disease | Hemoglobin |
| Myocardial Infarction | MAP4 |

Examples of autophagy-related proteins include ubiquitin-like proteins such as Atg-8 and Atg-12.

The aggregating protein may be a natural protein existing in nature, a modified protein obtained by artificially introducing an alteration or modification into a natural protein, or an artificial protein based on an artificially designed amino acid sequence.

### (3) Aggregate

As used herein, the term “aggregate” refers to an assembly of two or more aggregating proteins. Herein, so-called protein complexes are also encompassed by aggregates.

The aggregate may be a homoaggregate composed of the same kind of protein or a heteroaggregate composed of different kinds of proteins.

### (4) Deposition

As used herein, “deposition” of an aggregating protein on the surface of an organoid and/or inside an organoid refers to adhesion of the aggregating protein on the surface of the organoid and/or inside the organoid. The site of deposition may be a cell that forms an organoid or may be between a cell and an ECM.

### (5) Cell

A “cell” of interest in one or more embodiments of the present invention is a cell that forms an organoid.

The cell may be any cell derived from a multicellular organism. It is particularly an animal-derived cell, more particularly a mammalian-derived cell. Examples include rodents such as mice, rats, hamsters, and guinea pigs; domestic animals or pets such as dogs, cats, rabbits, cattle, horses, sheep, and goats; and primates such as humans, rhesus monkeys, gorillas, and chimpanzees. Particularly used are human-derived cells.

The type of cell is not limited. Examples thereof include a cell derived from a biological tissue, a cell derived from a cell derived from a biological tissue, a stem cell, or a cell differentiated from a stem cell or a progenitor cell thereof.

The term “biological tissue” refers to various tissues constituting a living body of an organism. Examples thereof include epithelial tissue, connective tissue, muscle tissue, and nerve tissue.

The term “stem cell” refers to a cell having the ability to differentiate into various cells and the ability to self-renew. Examples thereof include adult stem cells and pluripotent stem cells.

The term “adult stem cell” refers to a stem cell that is present in each tissue of an adult, is not yet terminally differentiated, and has a certain degree of multipotency, and is also referred to as a somatic stem cell or a tissue stem cell. Examples thereof include mesenchymal stem cells, neural stem cells, intestinal epithelial stem cells, hematopoietic stem cells, hair follicle stem cells, pigment stem cells, and cancer stem cells.

The term “pluripotent stem cell” refers to a cell that has multipotency (pluripotency) capable of differentiating into all kinds of cells constituting a living body and can continue to proliferate unlimitedly while maintaining pluripotency in culture in vitro under appropriate conditions. Examples thereof include embryonic stem cells (ES cells), embryonic germ stem cells, germ line stem cells, and induced pluripotent stem cells (iPS cells).

Furthermore, a neuronal cell obtained by further inducing differentiation of a neural progenitor cell obtained by inducing differentiation of an iPS cell derived from an Alzheimer's disease (AD) patient can be used for the formation of an organoid as a cell that produces and secretes an amyloid β protein such as amyloid β42 protein.

### (6) Extracellular Matrix

In this specification, a substrate in which cells are embedded is referred to as an extracellular matrix. The extracellular matrix may be any gel-state substrate in which cells can be embedded, and may be an animal-derived mixed protein represented by Matrigel, more particularly a polymer-derived substrate such as collagen, fibronectin, entactin, laminin, vitronectin, or an artificial protein.

### (7) Medium

In one or more embodiments of the present invention, the term “medium” refers to a liquid or solid substance prepared for culturing cells. In principle, the medium contains components essential for the growth and/or maintenance of cells at a necessary minimum amount or more. The medium may be either a basal medium or a specialized cell culture medium.

The term “basal medium” refers to a medium serving as a basis for various media for animal cells. The medium alone can be used for culture, or various culture additives can be added to prepare a medium specific to various cells according to the purpose (specialized cell culture medium). The basal medium is not limited, and examples include Neurobasal (registered trademark) medium, BME medium, BGJb medium, CMRL1066 medium, Glasgow MEM medium, Improved MEM Zinc Option medium, IMDM medium (Iscove's Modified Dulbecco's Medium), Medium 199 medium, Eagle MEM medium, αMEM medium, DMEM medium (Dulbecco's Modified Eagle's Medium), Ham F10 medium, Ham F12 medium, RPMI 1640 medium, Fischer's medium, and mixed media thereof (e.g., DMEM/F12 medium). In addition, a medium used for culturing human iPS cells or human ES cells can also be suitably used.

The “specialized cell culture medium” refers to a medium prepared so as to be optimal for the culture of specific cells by adding various supplements to a basal medium as described above, or a medium prepared so as to be capable of inducing differentiation into specific cells. Examples include neural cell culture media commercially available from various manufacturers. A specific example is “M medium”, which is prepared as follows: to a neuronal cell culture medium from Sumitomo bakelite prepared by adding a culture supernatant of primary astroglial cells cultured in a nutrient medium and serum albumin to a basal medium prepared by adding insulin and transferrin to DMEM/F12 (5:5), NGF2.5S and BDNF are further added as supplements. Another example is a medium for culturing pluripotent stem cells such as human iPS cells and human ES cells.

The medium may be either a serum-containing medium or a medium not containing serum (i.e., a serum-free medium).

### (8) Label

As used herein, the term “labeling” refers to modifying a target substance in order to identify the target substance. The labeling facilitates and ensures detection, selection, or the like of the target substance.

Labeling is performed according to the type of the target substance. In the present specification, since an aggregating protein is a target substance to be detected, the type thereof is a protein. Accordingly, the label may be any labeling means capable of directly or indirectly labeling the protein. Examples of direct labeling include a labeling method in which a labeling substance is bound to an aggregating protein, and a labeling method in which an aggregating protein is expressed as a fusion protein with a labeling peptide. Examples of indirect labeling include methods in which an antibody or active fragment thereof that specifically recognizes and binds to an aggregating protein is labeled directly or indirectly via a secondary antibody. The method of binding a labeling substance is particularly used.

Examples of labeling substances used for labeling proteins include, but are not limited to, optical labels.

As used herein, the term “optical label” refers to a label made of a substance that emits visible light, near-infrared light, or near-ultraviolet light, such as a fluorescent substance or a luminescent substance.

The “fluorescent substance” is a substance having a property of becoming an excited state by absorbing excitation light having a specific wavelength and emitting fluorescence when returning to the original ground state. The fluorescent substance includes both fluorescent dyes and fluorescent proteins.

Examples of the “fluorescent dyes” include quantum dots, FITC, Texas, Texas Red (registered trademark), Alexa Fluor 405, Alexa Fluor 488, Alexa Fluor 647, Alexa Fluor 700, Pacific Blue, DyLight 405, DyLight 550, DyLight 650, PE-Cy5 (phycoerythrin-cyanin5), PE-Cy7 (phycoerythrin-cyanine 7), PE (phycoerythrin), PerCP (peridinin chlorophyll protein), PerCP-Cy5.5 (peridinin chlorophyll protein-cyanin5.5), cy3, cy5, cy7, FAM, HEX, VIC (registered trademark), JOE, ROX, TET, Bodipy493, NBD, TAMRA, Quasar (registered trademark) 670, Quasar (registered trademark) 705, APC (Allophycocyanin), Congo red, Thioflavin T, Thioflavin S, fluorescamine or derivatives thereof, fluorescein or derivatives thereof, azos, rhodamine or derivatives thereof, coumarin or derivatives thereof, pyrene or derivatives thereof, and cyanine or derivatives thereof. Quantum dots are particularly used.

“Quantum dot” (often referred to herein as “QD”) refers to a nanoscale semiconductor crystal that has optical properties based on quantum mechanics and emits fluorescence in the visible and near-infrared regions. In general, it has a diameter of 2 nm to 10 nm, is composed of about 10 to 50 atoms, and has excellent characteristics such as providing a large number of fluorescent colors depending on the particle size and being resistant to fluorescence fading, and therefore, its applications as a biosensing material and an imaging material for cells or animals have been increased. Examples of quantum dots include semiconductor quantum dots, particularly, Qdot (registered trademark) 525, Qdot545, Qdot565, Qdot585, Qdot605, Qdot655, Qdot705, and Qdot800 (all of which are from Thermo Fisher Scientific), which are core-shell type CdSe/ZnS quantum dots.

Examples of “fluorescent proteins” include GFP.

### (9) Test Substance

Examples of the test substance include nucleic acids, peptides, proteins, synthetic compounds, cell extracts, cell culture supernatants, plant extracts, and marine algae extracts.

### 1-3. Method

Each step of the method of this aspect will be described below.

### 1-3-1. “Coexistence Step”

The “coexistence step” is a step of allowing a labeled aggregating protein, a test substance, and an organoid to coexist in an aqueous solution to culture (incubate) the organoid. In this step, the labeled aggregating protein is aggregated and/or deposited on the surface of the organoid and/or inside the organoid by culture (incubation).

The aqueous solution used here may be, for example, a medium, physiological saline, or Ringer's solution.

For example, in a culture vessel (e.g., a plate such as a 96-well microplate), a labeled aggregating protein-containing solution diluted with a medium or the like and a test substance-containing solution diluted with a medium or the like are added to an organoid. Furthermore, an unlabeled aggregating protein-containing solution diluted with a medium or the like may be added. The addition of these solutions can be carried out simultaneously or successively. The order of additions may be in any order.

Alternatively, the organoid is cultured (incubated) in the presence of a labeled aggregating protein, and by this culture, the labeled aggregating protein is aggregated and/or deposited on the surface of the organoid and/or inside the organoid. To the organoid on and/or in which the labeled aggregating protein is aggregated and/or deposited in advance in this manner, a test substance-containing solution is added, so that the labeled aggregating protein, the test substance, and the organoid can be allowed to coexist.

In the culture vessel, the respective concentrations of the aggregating protein and the test substance in the aqueous solution (medium) relative to, for example, an organoid with a diameter of 1 mm or more may be, for example, as follows: the labeled aggregating protein, a final concentration of 5 to 100 nM (particularly 10 to 50 nM); the test substance, a final concentration of 1 to 100 ng/μL (particularly 10 to 50 ng/μL); the unlabeled aggregating protein, a final concentration of 5 to 100 μM (particularly 10 to 50 μM).

The conditions for the coexistence or culture (incubation) are not particularly limited as long as the labeled aggregating protein can be aggregated and/or deposited on the surface of the organoid and/or inside the organoid. The temperature is, for example, 25° C. to 40° C., 30° C. to 39° C., or 34° C. to 38° C. The time may be, for example, 30 minutes to 1200 hours (50 days), 12 hours to 96 hours (4 days), 24 hours (1 day) to 72 hours (3 days), or 36 hours to 48 hours (2 days).

The culture (incubation) may be static culture or a culture under a condition in which the medium flows (flow culture), and is particularly static culture.

### 1-3-2. Quantifying Step

The “quantifying step” is a step of quantifying, using the label attached to the added aggregating protein as an index, the aggregating protein aggregated and/or deposited on the surface of the organoid and/or inside the organoid, the organoid being obtained in the coexistence step, over time (e.g., every 1 to 50 days, particularly every 3 to 40 days) either after culture (incubation) or while the organoid is maintained (while culture is continued). In particular, the quantifying can be performed for at least 7 days without using a genetically modified cell. In this step, aggregates/deposits of the aggregating protein on the surface of the organoid and/or inside the organoid are quantitatively detected.

For example, when the aggregating protein is labeled with an optical labeling substance such as a quantum dot, the aggregates/deposits may be detected by fluorescence observation using a fluorescence microscope. If fluorescence is observed on the surface of the organoid and/or inside the organoid, aggregates/deposits can be identified as being formed. The fluorescence intensity is then quantified, and the presence or absence of aggregates/deposits is quantitatively detected, for example, by comparison of fluorescence intensity values with negative controls.

Specifically, the quantifying step can include a step of capturing an image of the organoid, and a step of calculating the ratio of the area of a fluorescent region from the label to an organoid surface area as the aggregating protein aggregated and/or deposited on the surface of the organoid and/or inside the organoid, using fluorescence from the label such as a quantum dot in the image obtained in the capturing step as an index.

For example, when a quantum dot-modified amyloid β protein (e.g., amyloid β40 protein) and an unlabeled amyloid R protein (e.g., amyloid β42 protein) are added to a cerebral organoid, the cerebral organoid is irradiated with light, and aggregates/deposits containing the unlabeled amyloid β protein and the quantum dot-modified amyloid β protein on the surface of the cerebral organoid and/or inside the cerebral organoid are visualized by the quantum dots.

The area of the fluorescent region is then calculated from image acquisition of the cerebral organoid followed by image analysis for fluorescence due to the quantum dots. Since the area of the fluorescent region positively correlates with the amount of aggregated/deposited aggregating protein, whether or not the added test substance has an aggregation-suppressing activity or aggregation-promoting activity on the aggregating protein is evaluated, for example, by comparing the ratio of the area of the fluorescent region to the surface area of the cerebral organoid with a negative control (e.g., in the absence of the test substance) and/or a positive control (e.g., in the presence of a known aggregation-suppressing active substance or aggregation-promoting active substance).

For example, an image of the organoid is captured, and an organoid area in the image obtained by the capturing is calculated (organoid surface area).

Next, the area of the fluorescent region of the aggregating protein in the image is calculated (deposition area of aggregating protein).

The deposition rate (%) of the aggregating protein on and/or in the organoid can be calculated by the following formula (1):

$$( Deposition area of aggregating protein / organoid surface area ) \times 100 \qquad ( 1 )$$

Furthermore, for example, the function of the test substance to suppress the deposition of the aggregating protein can be quantified as a deposition suppression rate (%) and can be calculated by the following formula (2):

$$Deposition suppression rate ( \% ) of aggregating protein = ( deposition rate ( \% ) of aggregating protein in negative control ) - ( deposition rate ( \% ) of aggregating protein when test substance is added ) \qquad ( 2 )$$

The larger the deposition suppression rate (%) calculated by Formula (2) is, the greater the deposition suppression effect of the test substance is.

Thus, when the ratio of the area of the fluorescent region to the surface area of the organoid is lower than that of the negative control, the added test substance can be determined to have an aggregation-suppressing activity. When the ratio of the area of the fluorescent region to the surface area of the organoid is higher than that of the negative control, the added test substance can be determined to have an aggregation-promoting activity. When the ratio of the area of the fluorescent region to the surface area of the organoid is equal to or less than that of the positive control (e.g., in the presence of a known aggregation-suppressing active substance), the added test substance can be determined to have an aggregation-suppressing activity. When the ratio of the area of the fluorescent region to the surface area of the organoid is equal to or higher than that of the positive control (e.g., in the presence of a known aggregation-promoting active substance), the added test substance can be determined to have an aggregation-promoting activity.

### 1-4. Effect

According to the method of this aspect, effectiveness as a search tool for a drug useful for treatment or prevention of various diseases caused by aggregating proteins, including amyloidosis such as AD and Parkinson's disease, in a state closer to a biological environment can be expected.

### 2. Method for Producing Organoid

### 2-1. Overview

A second aspect of one or more embodiments of the present invention is a method for producing an organoid. The method of this aspect includes a step of forming a spheroid by culturing a cell, a step of embedding the spheroid obtained in the spheroid forming step in a gel, and a step of culturing the spheroid embedded in the gel embedding step in the gel. The method of this aspect can provide an organoid usable for the selection (screening) of a candidate substance that exhibits an aggregation-suppressing activity or aggregation-promoting activity on an aggregating protein in the method of the first aspect.

### 2-2. Method

Each step of the method of this aspect will be described below.

### 2-2-1. Spheroid Forming Step

The “spheroid forming step” is a step of forming a spheroid such as an embryoid body by culturing a cell.

The shape of a culture vessel used in this step is not particularly limited, and examples thereof include culture vessels having a dish shape, a flask shape, a well shape, a bag shape, a spinner flask shape, and the like.

In the culture vessel, the seeding concentration of the cell in a medium (cell concentration at the start of culture) can be adjusted as appropriate, and may be, for example, 0.01×10⁵ cells/mL or more, more particularly 0.1×10⁵ cells/mL or more, more particularly 1×10⁵ cells/mL or more, and 20×10⁵ cells/mL or less, more particularly 10×10⁵ cells/mL or less.

The temperature condition for culture is, for example, 25° C. to 40° C., 30° C. to 39° C., or 34° C. to 38° C.

On the other hand, the culture time is, for example, 10 hours or more, particularly 12 hours or more, more particularly 20 hours or more, and 300 hours or less, particularly 200 hours or less, more particularly 100 hours or less. When the culture time is too long, exceeding 100 hours, the binding between cells becomes too strong, and the cells are not successfully filled in a gel in the subsequent in-gel culturing step of the spheroid embedded in the gel.

The culture may be static culture or a culture under a condition in which the medium flows (flow culture), and is particularly static culture.

In this step, for example, an embryoid body may be formed during the process of inducing an iPS cell into a neuronal cell, or a neuronal cell embryoid body may be formed after the embryoid body formation from an iPS cell.

When an embryoid body is formed during the process of inducing an iPS cell into a neuronal cell, first, the iPS cell is cultured in a neural progenitor cell induction medium (e.g., a medium (hereinafter referred to as “NIMS medium”) prepared by adding STEMdiff™ SMADi Neural Induction Supplement to STEMdiff™ Neural Induction Medium), and induced to differentiate into a neural progenitor cell. At this time, a ROCK inhibitor may be added to the medium.

ROCK inhibitors are defined as substances that inhibit the kinase activity of Rho-kinase (ROCK: Rho-associated protein kinase), and examples thereof include Y-27632 (4-[(1R)-1-aminoethyl]-N-pyridin-4-ylcyclohexane-1-carboxamide or salts thereof (e.g., dihydrochloride)), H-1152 ((S)-(+)-2-methyl-1-[(4-methyl-5-isoquinolinyl)sulfonyl]-hexahydro-1H-1,4-diazepine or salts thereof (e.g., dihydrochloride)), Fasudil/HA1077 (1-(5-isoquinolinesulfonyl)homopiperazine or salts thereof (e.g., dihydrochloride)), Wf-536 ((+)-(R)-4-(1-aminoethyl)-N-(4-pyridyl)benzamide monohydrochloride), Y39983 (4-[(1R)-1-Aminoethyl]-N-1H-pyrrolo[2,3-b]pyridin-4-ylbenzamide dihydrochloride), SLx-2119 (2-[3-[4-(1H-indazol-5-ylamino)-2-quinazolinyl]phenoxy]-N-(1-methylethyl)-acetamide), Azabenzimidazole-aminofurazans, DE-104, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, and VAS-012.

The final concentration of the ROCK inhibitor in the medium is, for example, 5 μM or more, particularly 20 μM or more, and 100 μM or less, particularly 50 μM or less.

Next, the obtained neural progenitor cell is expanded in a neural progenitor cell culture medium (e.g., STEMdiff™ Neural Progenitor Medium). This neural progenitor cell is cultured in a neuronal cell culture medium (e.g., a mixed medium (hereinafter referred to as “NbM medium”) of Neuro basal Medium minus phenol red (Thermo Fisher Scientific) with B-27 supplement (Thermo Fisher Scientific) and Penicillin-Streptomycin Mixed Solution (Nacalai Tesque)) for the predetermined culture time mentioned above to form an embryoid body while inducing differentiation into a neuronal cell. A hedgehog protein (e.g., human recombinant sonic hedgehog (SHH, manufactured by Veritas)) is further added to the medium as an essential component. The final concentration of the hedgehog protein in the medium is, for example, 1 ng/mL or more, particularly 10 ng/mL or more, and 200 ng/mL or less, particularly 100 ng/mL or less.

On the other hand, when a neuronal cell embryoid body is formed after the embryoid body formation from an iPS cell, the iPS cell is cultured in an embryoid body formation medium (e.g., EB Formation medium in STEMdiff™ Cerebral Organoid Kit) for the predetermined culture time mentioned above to form an iPS cell embryoid body. Thereafter, the iPS cell embryoid body is cultured in a neuronal cell differentiation-inducing medium (e.g., a medium containing GMEM (Thermo Fisher Scientific), 5% KSR (Thermo Fisher Scientific), and AGN193109 (Sigma-Aldrich Japan)) and induced to differentiate into a neuronal cell, whereby the neuronal cell embryoid body can be obtained.

Whether or not differentiation induction to the neuronal cell has been achieved can be determined using a neural progenitor cell marker and/or a neuronal cell marker as an index. Examples of the neural progenitor cell marker include Nestin. Examples of the neuronal cell marker include MAP2.

The neural progenitor cell marker and/or the neuronal cell marker can be detected by any detection method in the technical field. Examples of methods of detecting expressed markers include, but are not limited to, flow cytometry. In flow cytometry using a fluorescent-labeled antibody, when a cell emitting stronger fluorescence is detected as compared with a negative control (isotype control), the cell is determined to be “positive” for the marker. The percentage of cells displaying positivity for fluorescent-labeled antibodies analyzed by flow cytometry is sometimes described as the positive rate. In addition, any antibody known in the technical field can be used as the fluorescent-labeled antibody, and examples thereof include, but are not limited to, antibodies labeled with fluorescein isothiocyanate (FITC), phycoerythrin (PE), allophycocyanin (APC), and the like.

In addition, the neural progenitor cell marker and/or the neuronal cell marker can be detected by an immunostaining reaction. For example, cells or embryoid bodies in a culture vessel are reacted with a primary antibody and then with a fluorescent-labeled secondary antibody, and visually fluorescing cells or embryoid bodies are detected by fluorescence microscopy. For example, the ratio of cells or embryoid bodies stained with a fluorescent-labeled secondary antibody to cells or embryoid bodies stained with DAPI (cell nucleus marker) can be used as the positive rate. As the fluorescent-labeled secondary antibodies, any antibodies known in the technical field can be used, and examples thereof include, but are not limited to, antibodies labeled with Alexa Fluor488 or the like.

For example, when the proportion (ratio) of cells or embryoid bodies positive for a neural progenitor cell marker is, for example, 10% or less, particularly 5% or less, and/or the proportion (ratio) of cells or embryoid bodies positive for a neuronal cell marker is 70% or more, particularly 80% or more, it can be determined that differentiation induction to neuronal cells has been significantly achieved.

### 2-2-2. Gel Embedding Step

In this step, the spheroid obtained in the spheroid forming step is embedded in a gel. For example, the spheroid is embedded in a gel such as Matrigel. Examples of the embedding method include a method in which a parafilm is applied to a culture plate, a depression is formed with a tip attachment portion or the like, the spheroid is placed in the depression, a gel is added dropwise thereto, the resultant is allowed to stand still for, for example, 10 minutes to 720 minutes (particularly 30 to 180 minutes), and then the gel in which the spheroid is embedded is collected in a new culture plate.

### 2-2-3. In-Gel Culturing Step

In this step, the spheroid embedded in the gel embedding step is cultured in the gel.

The spheroid embedded in the gel is subjected to shaking culture in a culture vessel containing a medium. For example, shaking culture is performed while being rotated at 10 to 200 rpm (particularly 30 to 150 rpm) in a Bioshaker.

The temperature condition for culture is, for example, 25° C. to 40° C., 30° C. to 39° C., or 34° C. to 38° C.

On the other hand, the culture time is, for example, 14 days or more, particularly 21 days or more, and 364 days or less, particularly 280 days or less, so that the cells sufficiently infiltrate into and fill the gel to form an organoid.

In the organoid production, it is important to know that the cells from the spheroid embedded in the gel infiltrate into and fill the gel, in order to know the maturity of the organoid and the production process. In this regard, it has been found that in this in-gel culturing step, a cell migration protein is secreted from the cells, and the concentration of the cell migration protein in a culture supernatant correlates with the cell infiltration state in the gel.

Therefore, the in-gel culturing step particularly includes measuring the concentration of the cell migration protein in the culture supernatant.

Examples of the cell migration protein include CCL2 (monocyte chemotactic protein).

The concentration of the cell migration protein in the culture supernatant can be measured by an immunological measurement method such as ELISA.

For example, when the concentration of CCL2 in the culture supernatant is 500 μg/ml or less, particularly 200 μg/ml or less, it can be determined that the filling of the cells in the gel is completed and the organoid is formed.

### 3. Organoid and Use Thereof

### 3-1. Overview

A third aspect of one or more embodiments of the present invention relates to an organoid produced by the method for producing an organoid according to the second aspect, and use thereof.

Specifically, the organoid of this aspect is an organoid with an aggregated and/or deposited aggregating protein, including an organoid produced by the method for producing an organoid according to the second aspect and an aggregating protein aggregated and/or deposited on the surface of the organoid and/or inside the organoid. By quantifying the aggregating protein aggregated and/or deposited on the surface of the organoid and/or inside the organoid by the method of the first aspect using the organoid with an aggregated and/or deposited aggregating protein, a candidate substance that actually exhibits an aggregation-suppressing activity or aggregation-promoting activity on the aggregating protein can be selected from among candidate substances added to the organoid.

### 3-2. Preparation of Organoid with Aggregated and/or Deposited Aggregating Protein

The produced organoid is cultured (incubated) in the presence of an aggregating protein. By this culture, the aggregating protein is aggregated and/or deposited on the organoid surface and/or in the organoid.

Specifically, in a culture vessel, the aggregating protein is added to the organoid having a diameter of, for example, 1 mm or more at a final concentration of 5 to 100 nM (particularly 10 to 50 nM), and cultured.

The culture conditions are not particularly limited as long as the aggregating protein can be aggregated and/or deposited on the organoid surface and/or in the organoid. The temperature is, for example, 25° C. to 40° C., 30° C. to 39° C., or 34° C. to 38° C. The time may be, for example, 30 minutes to 1200 hours (50 days), 12 hours to 96 hours (4 days), 24 hours (1 day) to 72 hours (3 days), or 36 hours to 48 hours (2 days).

The culture may be static culture or a culture under a condition in which the medium flows (flow culture), and is particularly static culture.

The organoid with an aggregated and/or deposited aggregating protein can gradually increase the amount of aggregating protein deposition as the number of days of culture increases while being maintained, and can be maintained for at least 8 days.

The aggregating protein is particularly labeled with an optical labeling substance such as a quantum dot. With this label, by quantifying the aggregating protein aggregated and/or deposited on the surface of the organoid and/or inside the organoid using the label as an index, a candidate substance that exhibits an aggregation-suppressing activity or aggregation-promoting activity on the aggregating protein can be selected from among candidate substances added to the organoid.

### 3-3. Use of Organoid with Aggregated and/or Deposited Aggregating Protein (Method for Quantifying Deposition of Aggregating Protein on and/or in Organoid)

According to the method of the first aspect, by culturing (incubating) the organoid with an aggregated and/or deposited aggregating protein in the presence of test substances, a candidate substance that exhibits an aggregation-suppressing activity or aggregation-promoting activity on the aggregating protein can be selected from among the test substances added to the organoid.

After the culturing, the aggregating protein aggregated and/or deposited on the organoid surface and/or in the organoid in the culture is quantified using the label attached to the added aggregating protein as an index. In this way, aggregates/deposits of the aggregating protein on the organoid surface and/or in the organoid are quantitatively detected.

For example, when the aggregating protein is labeled with an optical labeling substance such as a quantum dot, the aggregates/deposits may be detected by fluorescence observation using a fluorescence microscope in accordance with “1-3-2. Quantifying step” of the method of the first aspect.

Specifically, an image of the organoid is captured, and an organoid area in the image obtained by the capturing is calculated (organoid surface area).

Next, the area of the fluorescent range of the aggregating protein in the image is calculated (deposition area of aggregating protein).

The deposition rate (%) of the aggregating protein on and/or in the organoid can be calculated by the following formula (1):

$$( Deposition area of aggregating protein / organoid surface area ) \times 100 \qquad ( 1 )$$

Furthermore, for example, the function of the test substance to suppress the deposition of the aggregating protein can be quantified as a deposition suppression rate (%) and can be calculated by the following formula (2):

$$Deposition suppression rate ( \% ) of aggregating protein = ( deposition rate ( \% ) of aggregating protein in negative control ) - ( deposition rate ( \% ) of aggregating protein when test substance is added ) \qquad ( 2 )$$

The larger the deposition suppression rate (%) calculated by Formula (2) is, the greater the deposition suppression effect of the test substance is.

According to the method for quantifying the deposition of an aggregating protein on and/or in an organoid of this aspect, effectiveness as a search tool for a drug useful for treatment or prevention of various diseases caused by aggregating proteins, including amyloidosis such as AD and Parkinson's disease, in a state closer to a biological environment can be expected.

### EXAMPLES

Hereinafter, one or more embodiments of the present invention will be described in more detail with reference to Examples, but the technical scope of one or more embodiments of the present invention is not limited to these Examples.

Reagents used in Examples were as follows.

### <Reagents>

STEMdiff™ Neural Progenitor Medium (NPM) (STEMCELL/5833)
Neuro basal medium minus phenol red (Gibco/12348017)
hESC-Qualified Matrigel (hES-matrigel) (Corning/354277)
Accutase (STEMCELL/7920)
DMEM/F-12 (Gibco/21041025)
Y-27632 (nacalao/08945-84)
PBS (Gibco/10010-031)
B-27 (Gibco/17504044)
Penicillin-Streptomycin (Nacalai/26253-84)
Amyloid β-Protein human 1-42 (Peptide Institute, Inc./4349-v)
1,1,1,3,3,3-Hexafluoro-2-2propanol (HFIP) (Tokyo Chemical Industry/H0424)
Dimethyl sulfoxide (DMSO) (FUJIFILM Wako Pure Chemical Industries, Ltd./046-21981)
QD reagent (described below)
Plant material solution (100 mg/mL solution: described below)
Physiological saline (saline) (Otsuka Pharmaceutical Co., Ltd.)
1 mL syringe
0.2 μm filter
Preparation of NbM medium

NbM medium was prepared by adding 1 mL of B-27 (Gibco/17504044) and 500 μL of Penicillin-Streptomycin (Nacalai/26253-84) to 50 mL of Neuro basal medium minus phenol red (Gibco/12348017).

### [Example 1] Preparation of Cerebral Organoid

### (1) Culture and Passage of iPS Cells

Human iPS cells, HPS08554 (RIKEN BRC), were seeded on a cell culture dish coated with Matrigel (Corning), Vitronectin (Life Technologies Japan, Ltd.), iMatirx (Takara, T303), or fibronectin (Recombinant HUMAN Fibronectin GMP Protein, CF Summary). mTeSR1 (STEMCELL Technologies), TeSR2 (STEMCELL Technologies), Essential 8™ (Life Technologies Japan, Ltd.), or StemFit was used as a medium for maintenance culture.

As a cell detachment agent for passage, Accutase (Life Technologies Japan, Ltd.) or GCDR (STEMCELL Technologies) was used for culture on Matrigel or Vitronectin, 0.05% EDTA (ethylenediaminetetraacetic acid) solution was used for culture on fibronectin, and TrypLE Select (Life Technologies Japan, Ltd.) or TrypLE Express was used for culture on iMatirx.

Only at the time of cell seeding, Y-27632 (Wako Pure Chemical Industries, Ltd.) was added to the medium to a concentration of 10 μM. Medium change was performed daily or every other day. In the experiment, human iPS cells that had undergone 50 passages or less were used.

### (2) Preparation of Neural-Inducing Embryoid Bodies (EBs)

After thawing, human iPS cells that had undergone at least two passages were treated with a detachment agent for 5 to 15 minutes, detached from the culture substrate, and dispersed into single cells by pipetting using a pipetteman. The single cells were suspended in a medium prepared by adding STEMdiff™ SMADi Neural Induction Supplement (hereinafter referred to as “NIMS medium”) to STEMdiff™ Neural Induction Medium containing Y-27632 (Wako Pure Chemical Industries, Ltd.) at a final concentration of 10 μM, and the cells were seeded on PLO/Laminin coat or Matrigel (registered trademark) (Corning (registered trademark)) and cultured in a 5% CO₂ and 37° C. environment.

After confirmation of cell adhesion, the NIMS medium was completely replaced every day. On day 7, adherent cells were detached with Accutase, and the detached cells were expanded in STEMdiff™ Neural Progenitor Medium on PLO/laminin coat or Matrigel for three passages. The cells were suspended in NbM plus Shh medium prepared by adding SHH (human recombinant sonic hedgehog (Veritas)) to a mixed medium (hereinafter referred to as “NbM medium”) of 50 mL of Neuro basal Medium minus phenol red (Thermo Fisher Scientific), 1 mL of B-27 supplement (Thermo Fisher Scientific), and Penicillin-Streptomycin Mixed Solution (Nacalai Tesque) to a final concentration of 1 ng/mL, thereby preparing a cell suspension of 4×10⁵ cells/mL. The suspension was seeded in 0.1 mL aliquots in a U-bottomed 96-well plate, and then cultured for 24 hours in a 5% CO₂ and 37° C. environment to prepare neural-inducing EBs.

Alternatively, human iPS cells cultured in mTeSR1 (STEMCELL Technologies) were treated with GCDR to form single cells, and a cell suspension of 9×10⁴ cells/mL was prepared. The suspension was seeded in 0.1 mL aliquots in a U-bottomed 96-well plate, and cultured in a 5% CO₂ and 37° C. environment for 5 days while the medium was replaced with EB Formation medium in STEMdiff™ Cerebral Organoid Kit every two days. Thereafter, the medium was replaced with Induced Medium, and EBs formed from the iPS cells by performing culture for another 2 days were neurally induced to prepare neural-inducing EBs.

### (3) Gel-Embedded Culture of Neural-Inducing EBs

EBs 2 days after hedgehog signal addition (on Day 2) and EBs 5 days after hedgehog signal addition (on Day 5) were each carefully collected with a wide-bore pipette tip so as not to collapse, and 12 EBs were embedded in Matrigel. Although there are multiple embedding methods, in this Example, a parafilm was applied to a 48-well plate, the required number of depressions were formed using a tip attachment portion or the like, 12 EBs were placed in the depressions, and an excess of medium was removed. Thereafter, 15 μL of Matrigel was added dropwise, and the plate was allowed to stand in an incubator for about 0.5 to 2 hours. A fresh NbM medium was added dropwise to a 6-well plate for suspension, and the Matrigel was recovered using a tip with its end cut with scissors so as not to collapse.

Each of the EBs embedded in the Matrigel was cultured in a 5% CO₂ and 37° C. environment while being rotated at 90 rpm in a Bioshaker. During the gel-embedded culture period, half of the NbM medium was replaced every three days.

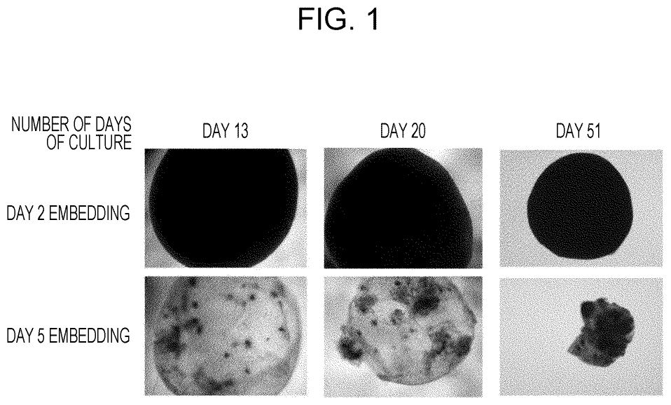

As shown in FIG. 1, as a result of confirming how organoid-like tissue structures were formed, a good tissue structure was formed from Day 2 EBs, whereas in the case of Day 5 EBs, the cells did not sufficiently infiltrate into and fill the gel.

### (4) Gel-Embedded Culture and Quantification of CCL2 in Culture Supernatant

Each of the EBs embedded in the Matrigel was cultured in a 5% CO₂ and 37° C. environment while being rotated at 90 rpm using a Bioshaker. The cells in the gel actively infiltrate and proliferate to fill the gel space. Thus, the medium was collected at the in-gel culturing initial stage (unfilled stage: D0), the in-gel culturing growth stage (unfilled stage: D7), the in-gel cell-filling stage (filling stage: D14), and the in-gel cell-filling stage (filling stage: D21), and the amount of cytokine secretion (CCL2) in the culture supernatant was quantified by ELISA.

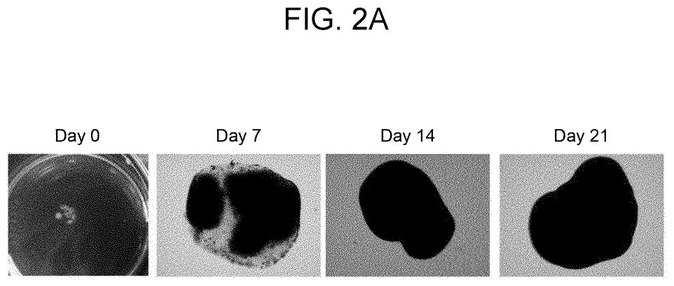

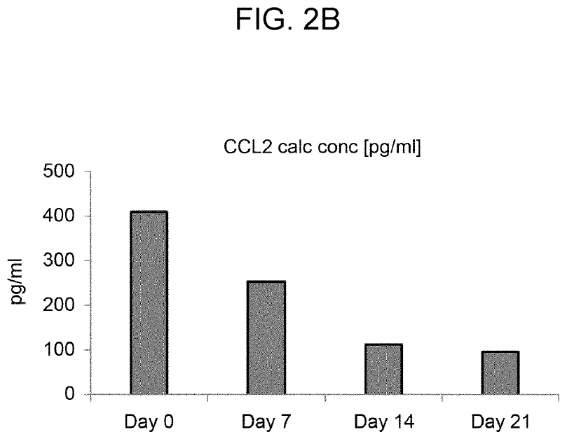

The results are shown in FIGS. 2A and B. It has been found that there is a correlation between the state of cell infiltration in the gel (FIG. 2A) and the amount of CCL2 (monocyte chemotactic protein) secretion (FIG. 2B). It has been found that the amount of CCL2 secretion remains high while the cells are infiltrating into and filling the gel, decreases as the filling progresses, and is eventually stabilized at a certain level.

In this manner, by measuring the CCL2 level in the culture supernatant during the organoid-like tissue structure formation process, it is possible to confirm whether the cells have filled the gel.

### [Example 2] Preparation of Organoid with Visualized Aggregating Protein Deposition, and Method for Searching for Aggregating Protein Deposition Suppressor Using this Organoid

An end of a tip for 1000 μL was cut with sterilized scissors, and the organoid grown to a diameter of 1 mm or larger prepared in Example 1 was sucked so as not to collapse, and transferred to a U-bottom 96-well plate. Thereafter, the medium was removed from the U-bottom 96-well plate, and (i) 25 μL of a quantum dot-modified amyloid β protein (QD-AP) solution, (ii) 25 μL of an amyloid β42 protein (Aβ42) solution, and (iii) 50 μL of a test sample solution (ratio of (i):(ii):(iii)=1:1:2) were added dropwise to one well and cultured at 5% CO₂ and 37° C. for 24 hours (final concentrations: QD-Aβ 30 nM, artificial Aβ solution 10 μM, test samples (rosmarinic acid: 50 μM, Hypericum ascyron: 0.01 mg/well, Geranium yesoense: 0.01 mg/well, Bombax ceiba extract: 0.01 mg/well)).

The preparation of each solution was as follows.

### (i) Preparation of Solution (120 nM QD-Aβ Solution)

Quantum dot-modified amyloid β protein (QD-Aβ) was prepared by modifying Aβ with quantum dots (QDs) by the following procedure.

125 μL of 8 μM Qdot(QD)™ 605 ITK™ amino (PEG) Quantum Dots were placed in two 1.5 mL tubes and centrifuged at 10000×g at 4° C. for 1 minute. Each supernatant was transferred to a centrifugal concentration tube, and 4500 μL of PBS was added. The two tubes were centrifuged at 4° C. and 3800×g until the total volume of the two tubes reached approximately 50 μL or less, and the filtrate was discarded. After the tubes were replenished with PBS, the mixture was centrifuged again to 50 μL. The resulting QD solutions were combined to a total volume of about 180 μL. 20 μL of 10 mM sulfo-EMCS was added, and the mixture was allowed to stand at room temperature for 1 hour to prepare QD-EMCS. After the preparation of QD-EMCS, 20 μL of 100 mM K-glutamate was added to inactivate unreacted N-hydroxysuccinimide groups contained in the solution, and the mixture was allowed to stand at room temperature for 10 minutes.

About 800 μL of resin was placed in each of two desalting columns prepared, and centrifuged at 1000×g at 4° C. for 1 minute. Each column was loaded with 300 μL of PBSE and centrifuged at 1000×g at 4° C. for 1 minute. This process was performed twice to prepare desalting columns.

After 110 μL of the QD-EMCS was allowed to permeate into the center of two desalting columns, 15 μL of PBSE was added as a stacker. After centrifugation at 1000×g at 4° C. for 2 minutes, the filtrates of the two desalting columns (desalted QD-EMCS) were combined. 1.0 mM Cys-Aβ (20 μL/DMSO) was added to the obtained desalted QD-EMCS, mixed, and then allowed to stand at room temperature for 1 hour. Thereafter, in order to inactivate unreacted maleimide groups contained in the solution, 20 μL of 100 mM 2-mercaptoethanol was added, and the mixture was allowed to stand at room temperature for 10 minutes.

By the above operation, 145 μL of the filtrate was transferred to each of two centrifugal concentration tubes, 4500 μL of water was added, and the mixture was then centrifuged at 3800×g at 4° C. for 17 minutes. The filtrate was discarded, and the resulting solutions were combined to a total volume of approximately 140 μL.

The desalting columns were filled with water (300 μL) and centrifuged at 1000×g at 4° C. for 1 minute. This process was repeated twice. 70 μL of the filtrate was allowed to permeate into each of the desalting columns, and 15 μL of stacker water was added. The filtrates were centrifuged at 1000×g at 4° C. for 2 minutes to obtain the desired QD-Ap.

The synthesized QD-Ap solution (80 μM) was diluted to 120 nM with NbM medium and sonicated at 43 kHz at 25° C. for 5 minutes in an ultrasonic cleaner.

### (ii) Preparation of Solution (40 μM Artificial Aβ42 Solution)

To a reagent bottle of Aβ₁₋₄₂ 0.5 mg (manufactured by Peptide Institute, Inc.: Cat No. 4394v), 500 μL of 1,1,1,3,3,3-Hexafluoro-2-propanol (HFIP) solution was added, and after pipetting, the reagent bottle was sealed with a parafilm, allowed to stand in a clean bench at room temperature for 1 hour, and uncapped in the clean bench to volatilize HFIP. After confirming that all HFIP had volatilized, 107 μL of DMSO was added, and the mixture was dissolved over 15 min. The solution was dispensed into tubes in 10 μL aliquots and cryopreserved at −80° C. After thawing the cryopreserved 1 mM Aβ42 solution, the solution was pipetted 20 or more times and diluted 25-fold with NbM medium to prepare a 40 μM Aβ solution.

(iii) Preparation of Test Sample-Containing Solutions
a. Preparation of Low Molecular Compound Solution (100 μM)

Rosmarinic acid (hereinafter referred to as “RA”) was dissolved in DMSO to prepare a 1 mM RA solution, which was then diluted to 100 μM with NbM medium to prepare a 100 μM RA solution.

b. Preparation of Plant Extracts (0.02 mg/Well) Preparation of Plant (Hypericum Ascyron and Geranium Yesoense) Extracts (Final Extract Concentration at the Time Of Testing: 0.01 mg/Well)

The leaves and stems of Hypericum ascyron and Geranium yesoense cultivated in Shiranuka Town, Hokkaido without pesticides for 3 months or more were collected, washed, and then dried at a constant temperature of 25° C. Subsequently, 1 g of the dried Hypericum ascyron and 1 g of the dried Geranium yesoense were each immersed in 30 mL of 90% EtOH and extracted by shaking at 28° C. in the dark for 20 hours at 150 rpm. The extracted liquids were centrifuged at 3,000 rpm for 20 minutes, after which the resultants were filtered through a cotton plug, further concentrated by centrifugation, and then freeze-dried. The dried products were dissolved in DMSO to prepare 100 mg/mL of Hypericum ascyron extract (hereinafter referred to as Hypericum ascyron) and Geranium yesoense extract, which were stored at −20° C. until use.

The material extracts were diluted 25-fold with NbM medium, and 40 μL of the diluted solutions were diluted with 960 μL of NbM medium, then filter-sterilized using a 0.2 μm filter to prepare 0.02 mg of material-containing solutions.

Additionally, 1 μL of DMSO solution and/or 150 mM physiological saline was diluted with 19 μL of NbM, and 3 μL of this diluted solution was further taken out to prepare 72 μL of a solution diluted with NbM.

The next day, the culture supernatant was completely removed so as not to suck the organoid, and after 150 μL of a fresh NbM medium was added, the cultured organoid was observed and photographed using a fluorescence microscope.

After photographing, the organoid was returned to the incubator again and cultured, and the observation and photographing using a fluorescence microscope were conducted in the same manner on subsequent days.

Preparation of Bombax ceiba extract (0.02 mg/well) Bombax ceiba obtained in Okinawa prefecture was washed and dried, after which 30 mL of 90% EtOH was added to 1 g of the dried product, and the mixture was extracted by shaking at 28° C. in the dark for 20 hours at 150 rpm. The extracted liquid was centrifuged at 3,000 rpm for 20 minutes, after which the resultant was filtered through a cotton plug, further concentrated by centrifugation, and then freeze-dried. This dried material was dissolved in DMSO to prepare 100 mg/mL of Bombax ceiba extract, which was stored at −20° C. until use.

The material extract in an amount of 1 μL was diluted with 19 μL of NbM medium, and 360 μL of NbM medium was added to 15 μL of the diluted solution. The mixture was filter-sterilized using a 0.2 μm filter. Additionally, 1 μL of DMSO solution and/or 150 mM physiological saline was diluted with 19 μL of NbM, and 3 μL of this diluted solution was further taken out to prepare 72 μL of a solution diluted with NbM. The next day, the culture supernatant was completely removed so as not to suck the organoid cultured in a mixed solution of solution (i), solution (ii), and solution (iii), and after 150 μL of a fresh NbM medium was added, the cultured organoid was observed and photographed using a fluorescence microscope. After photographing, the organoid was returned to the incubator again and cultured, and the observation and photographing using a fluorescence microscope were conducted in the same manner on subsequent days.

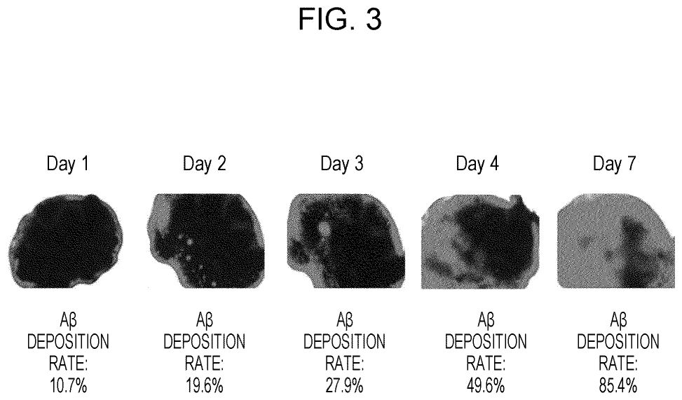

FIG. 3 shows how aggregation and deposition of Aβ proceed over time, as determined by observation and photographing.

As shown in FIG. 3, it has been confirmed that the organoid with visualized aggregating protein deposition can gradually increase the amount of aggregating protein deposition as the number of days of culture increases while maintaining the organoid, and can be observed for at least 8 days. From the above results, it has been found that an organoid with visualized aggregating protein deposition that mimics the rate of aggregating protein accumulation with aging in the human brain can also be produced.

### [Example 3] Method for Quantifying Deposition of Aggregating Protein on and/or in Organoid

Using a fluorescent microscope (BZ-X800 manufactured by Keyence Corporation), the cultured organoid in Example 2 was photographed in a bright field at high resolution with an exposure time of 1/500 s to 1/2000 s, or at high sensitivity with an exposure time of 1/2000 s to 1/5000 s, using a Z stack with 20 μm intervals. When using a 2× objective lens, one image showing the entire organoid was taken, and when using a 4× objective lens, one image with the center of the cultured organoid as the central position and three images including the outline of the cultured organoid were taken, obtaining a total of four images. Thereafter, in order to observe the deposition of quantum dot-modified amyloid β on the cultured organoid, red fluorescence was observed using a TRITC lens at high sensitivity with an exposure time of ¼ s to 1/8.5 s, and images were obtained according to the above procedure.

Each of the images obtained above was analyzed as follows. Specifically, the hybrid cell count function was used to read the organoid images used in the various tests, select the bright field, and then set the extraction selection to OFF. The threshold of the read bright-field images was set to 200, and the organoid area was calculated (organoid surface area).

Subsequently,
images of organoids with QD-Aβ/artificial Aβ added during organoid culture and no test specimens added;
images of negative control organoids with QD-Aβ/artificial Aβ and DMSO and/or physiological saline as a test sample added; and
images of organoids with rosmarinic acid (RA) or three kinds of plant extracts (Hypericum ascyron, Geranium yesoense, and Bombax ceiba extracts) added as test samples were read.

The black balance level in the image processing of fluorescence images from the various tests was adjusted. First, images serving as negative controls were read out, and whether the background and the fluorescent region of the aggregating protein deposited on and/or in the organoid was separable was confirmed, with the initial level of an image taken with the 2× objective lens at [50] and the initial level of an image taken with the 4× objective lens at [70]. When the background and the fluorescent region of the aggregating protein deposited on and/or in the organoid could not be separated at these black balance levels, the black balance level was varied in increments of 5 to set an optimal black balance level.

After combining the fluorescence image after applying the black balance and the bright-field organoid image, fluorescence was selected by the hybrid cell count function, and then one extraction was set. The detail mode of brightness was selected, the extraction setting range was set to 0-299, then after selecting an organoid, the blur filter was unchecked, the threshold value was set to 25, and the area of the fluorescent range of the aggregating protein was calculated (deposition area of aggregating protein).

The deposition rate (%) of the aggregating protein on and/or in the organoid was calculated by the following formula (1):

$$( Deposition area of aggregating protein / organoid surface area ) \times 100 \qquad ( 1 )$$

Whether the above test system was established was determined by whether the deposition rate (%) of the aggregating protein on and/or in an organoid with QD-Aβ/artificial Aβ added during organoid culture and no test specimens added and/or a negative control organoid with QD-Aβ/artificial Aβ and DMSO and/or physiological saline as a test sample added was in the range of 90% to 100%.

Furthermore, when this test system was established, by creating a group to which a test sample was also added simultaneously, it was possible to quantitatively analyze the suppression effect of the sample on aggregating protein deposition on and/or in the organoid. Specifically, the function of the test sample to suppress the deposition of the aggregating protein was able to be quantified as a deposition suppression rate (%), which was calculated using the following formula (2):

$$Deposition suppression rate ( \% ) of aggregating protein = ( deposition rate ( \% ) of aggregating protein in negative control ) - ( deposition rate ( \% ) of aggregating protein when test substance is added ) \qquad ( 2 )$$

The larger the deposition suppression rate (%) calculated by Formula (2) is, the greater the deposition suppression effect of the test sample is. The present evaluation technique can be applied to a technique for selecting a drug, a compound, a food, or a substance capable of preventing or delaying deposition of an aggregating protein on and/or in a cell or a tissue.

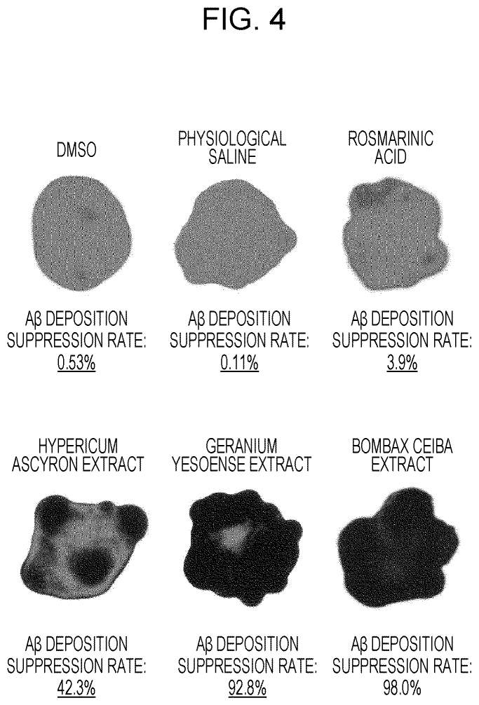

The results of the Aβ deposition suppression effect of each test sample in this Example are shown in FIG. 4.

As shown in FIG. 4, it was confirmed that the Geranium yesoense extract and the Bombax ceiba extract had an effect of suppressing the deposition of an aggregating protein on and/or in an organoid incomparable to those of DMSO, physiological saline, and rosmarinic acid. It was confirmed that the Aβ deposition suppression rates of DMSO, physiological saline, and rosmarinic acid were all less than 5.0%, but the Aβ deposition suppression rate of the Geranium yesoense extract was 92.8%, the Aβ deposition suppression rate of the Hypericum ascyron extract was 42.3%, and the Aβ deposition suppression rate of the Bombax ceiba extract was 98.0%. It follows from the foregoing that the Geranium yesoense extract and the Bombax ceiba extract can be used for preventing diseases caused by aggregating proteins.

### INDUSTRIAL APPLICABILITY

According to one or more embodiments of the present invention, a candidate substance that actually exhibits an aggregation-suppressing activity or aggregation-promoting activity on an aggregating protein can be selected from among candidate substances added to an organoid by quantifying the aggregated and/or deposited aggregating protein in the organoid. In particular, according to one or more embodiments of the present invention, candidate compounds for treating AD can be screened.

In addition, according to one or more embodiments of the present invention, an organoid that can be used for screening of a candidate compound useful for prevention or treatment of a disease can be produced. By quantifying the aggregating protein aggregated and/or deposited on the surface of the organoid and/or inside the organoid using the organoid produced according to one or more embodiments of the present invention, a candidate substance that actually exhibits an aggregation-suppressing activity or aggregation-promoting activity on the aggregating protein can be selected from among candidate substances added to the organoid.

All publications, patents, and patent applications cited herein are incorporated herein by reference in their entirety.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

1. A method for evaluating an aggregation-suppressing activity or aggregation-promoting activity of a test substance on an aggregating protein, the method comprising:
a step of allowing an aggregating protein labeled with a label, a test substance, and an organoid to coexist in an aqueous solution; and
a step of quantifying the aggregating protein aggregated and/or deposited on a surface of the organoid and/or inside the organoid using the label as an index.

2. The method according to claim 1, wherein the organoid is selected from the group consisting of a cerebral organoid, a cerebellar organoid, an inner ear organoid, a thyroid organoid, a thymus organoid, a T-cell matured lymphoid organoid, a cardiac muscle organoid, a lung organoid, a liver organoid, a pancreas organoid, a kidney organoid, a gastric gland organoid, a gut organoid, an epithelium organoid, an ovary organoid, a testis organoid, and a fused organoid including these organoids.

3. The method according to claim 1, wherein the aggregating protein is selected from the group consisting of amyloid β protein, tau protein, α-synuclein protein, prion protein, huntingtin protein, amylin protein, apolipoprotein A1, serum amyloid A protein, immunoglobulin light chain, MAβ4 protein, β2 microglobulin, TDP-43 protein, and cystatin C protein.

4. The method according to claim 1, wherein the label is an optical label.

5. The method according to claim 4, wherein the optical label is a quantum dot.

6. The method according to claim 1, wherein the step of quantifying comprises:
a step of capturing an image of the organoid; and
a step of calculating, as the aggregating protein aggregated and/or deposited on the surface of the organoid and/or inside the organoid, a ratio of an area of a fluorescent region from the label to a surface area of the organoid in the image.

7. The method according to claim 1, wherein the step of quantifying is performed over time while the organoid is maintained.

8. The method according to claim 1, wherein the step of quantifying is performed for at least 7 days without using a genetically modified cell.

9. A method for producing an organoid, comprising:
a step of forming a spheroid by culturing a cell for a culture time of 20 hours or more and 100 hours or less;
a step of embedding the spheroid in a gel; and
a step of culturing the spheroid embedded in the gel.

10. The method according to claim 9, wherein the step of culturing the spheroid in the gel comprises measuring a concentration of a cell migration protein in a culture supernatant.

11. The method according to claim 9, wherein the organoid is selected from the group consisting of a cerebral organoid, a cerebellar organoid, an inner ear organoid, a thyroid organoid, a thymus organoid, a T-cell matured lymphoid organoid, a cardiac muscle organoid, a lung organoid, a liver organoid, a pancreas organoid, a kidney organoid, a gastric gland organoid, a gut organoid, an epithelium organoid, an ovary organoid, a testis organoid, and a fused organoid including these organoids.

12. The method according to claim 9, wherein the cell is selected from the group consisting of an induced pluripotent stem cell (iPS cell), a neural progenitor cell, a mesenchymal stem cell, and a cell differentiated from these cells.

13. An organoid with an aggregated and/or deposited aggregating protein, comprising an organoid produced by the method according to claim 9 and an aggregating protein aggregated and/or deposited on a surface of the organoid and/or inside the organoid.

14. The organoid according to claim 13, wherein the organoid is maintained.
